(19) United States
(12) Patent Application Publication
(10) Pub. No.: US 2025/0270479 A1
(43) Pub. Date: Aug. 28, 2025

(54) Windshield Cleaning Composition And Method Of Use

(71) Applicant: Gilbert Valencia, Corpus Christi, TX (US)

(72) Inventor: Gilbert Valencia, Corpus Christi, TX (US)

(21) Appl. No.: 18/588,896

(22) Filed: Feb. 27, 2024

(51) Int. Cl. C11D 3/50; A61L 9/14; B60H 3/00; B60S 1/46; C11D 1/22; C11D 1/75; C11D 1/83; C11D 3/04; C11D 3/20; C11D 3/24; C11D 3/30; C11D 3/43

(52) U.S. Cl. CPC C11D 3/50; A61L 9/145; B60H 3/0092; C11D 1/83; C11D 3/044; C11D 3/2068; C11D 3/245; C11D 3/30; C11D 3/43; A61L 2209/16; B60S 1/46; C11D 1/22; C11D 1/75; C11D 2111/18

(57) ABSTRACT

A windshield cleaning composition for freshening air in a vehicle using a windshield washing system of the vehicle includes a windshield cleaning composition. The windshield cleaning composition comprises a solution that is to be added to a wiper fluid reservoir of a vehicle. The solution comprises water, a cleaning agent, an antifreeze agent, and an aroma compound. Dispensing of the solution onto a windshield of the vehicle positions the aroma compound to be drawn into a passenger compartment of the vehicle by a heating, cooling, and air conditioning system (HVACS) of the vehicle, thereby freshening the air of the vehicle.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to air freshening compositions and more particularly pertains to a new air freshening composition for freshening air in a vehicle using a windshield washing system of the vehicle. The confined space of a passenger compartment of a vehicle can retain obnoxious odors, which can be distracting or embarrassing to a driver.

### (2) Description of Related Art Including Information Disclosed Under 37 CFR 1.97 and 1.98

The prior art relates to air freshening compositions, which may be dispensed by devices positioned within a passenger compartment of a vehicle. The prior art also may comprise a fragrance or a solution of a fragrance that can be added to windshield washer solution.

### BRIEF SUMMARY OF THE INVENTION

An embodiment of the disclosure meets the needs presented above by generally comprising a windshield cleaning composition, which in turn comprises a solution that is to be added to a wiper fluid reservoir of a vehicle. The solution comprises water, a cleaning agent, an antifreeze agent, and an aroma compound. Dispensing of the solution onto a windshield of the vehicle positions the aroma compound to be drawn into a passenger compartment of the vehicle by a heating, cooling, and air conditioning system (HVACS) of the vehicle.

Another embodiment of the disclosure includes a windshield cleaning composition comprising a solution to be added to a wiper fluid reservoir of a vehicle. The solution comprises water, a cleaning agent, an antifreeze agent, and aroma compound. The cleaning agent comprises one or more of ammonia, 2-hexoxyethanol, lauryl dimethyl amine oxide, sodium dodecylbenzene sulfonate, isopropanolamine, butoxyethanol, perchloroethylene, tetrachloroethylene, ethanolamine, sodium hydroxide, or the like. The cleaning agent comprises from 0.2 to 5.0% by volume of the solution. The antifreeze agent comprises one or more of ethanol, isopropanol, ethylene glycol, propylene glycol, glycerol, 1,3 butanediol, 2-butanone, or the like. The antifreeze agent comprises from 0.5 to 50.0% by volume of the solution. The aroma compound comprises from 0.25 to 8.0% by volume of the solution. Dispensing of the solution onto a windshield of the vehicle positions the aroma compound to be drawn into a passenger compartment of the vehicle by the HVACS to freshen air in the passenger compartment.

Yet another embodiment of the disclosure includes a method of freshening air in a passenger compartment of a vehicle, which entails provision of a windshield cleaning composition, according to the disclosure above. Steps of the method include adding the solution to the wiper fluid reservoir of the vehicle, actuating the HVACS, actuating a windshield washing system of the vehicle, and drawing of the aroma compound into the passenger compartment by the HVACS to freshen the air in the passenger compartment.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWING(S)

FIG. 1 is a flow diagram for a method utilizing an embodiment of the disclosure.

### DETAILED DESCRIPTION OF THE INVENTION

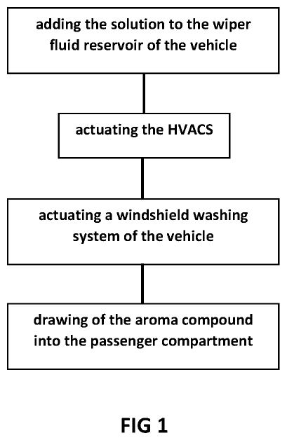

With reference now to FIG. 1, a new air freshening composition embodying the principles and concepts of an embodiment of the disclosure and generally designated by the reference numeral 10 will be described.

As best illustrated in FIG. 1, the windshield cleaning composition 10 generally comprises a windshield cleaning composition, which in turn comprises a solution that is to be added to a wiper fluid reservoir of a vehicle. The solution comprises water, a cleaning agent, an antifreeze agent, and an aroma compound. Dispensing of the solution onto a windshield of the vehicle positions the aroma compound to be drawn into a passenger compartment of the vehicle by a heating, cooling, and air conditioning system (HVACS) of the vehicle.

The antifreeze agent comprises one or more of ethanol, isopropanol, ethylene glycol, propylene glycol, glycerol, 1,3 butanediol, 2-butanone, or the like. The antifreeze agent comprises from 0.5 to 50.0% by volume of the solution. The antifreeze agent may comprise from 0.5 to 10.0% by volume of the solution.

The cleaning agent comprises one or more of ammonia, 2-hexoxyethanol, lauryl dimethyl amine oxide, sodium dodecylbenzene sulfonate, isopropanolamine, butoxyethanol, perchloroethylene, tetrachloroethylene, ethanolamine, sodium hydroxide, or the like. The cleaning agent comprises from 0.2 to 5.0% by volume of the solution. The cleaning agent may comprise from 0.5 to 2.5% by volume of the solution.

The aroma compound comprises from 0.25 to 8.0% by volume of the solution. The aroma compound may comprise from 3.3 to 5.0% by volume of the solution. There is a wide variety of aroma compounds anticipated by the present invention, such as, but not limited to, geranyl acetate, isoamyl acetate, pentyl pentanoate, benzyl acetate, methyl salicylate, geraniol, nerol, citronellal, citronellol, nerolidol, limonene, terpineol, eucalyptol, jasmone, cinnamaldehyde, ethyl maltol, vanillin, anisole, furaneol, menthol, or the like. The solution also may include a dye so that the solution is colored, thereby facilitating visualization of a level of the solution within the wiper fluid reservoir.

The windshield cleaning composition enables a method of freshening air in a passenger compartment of a vehicle, which entails provision of a windshield cleaning composition, according to the specification above. As is shown in FIG. 1, a first step of the method is adding the solution to the wiper fluid reservoir of the vehicle. A second step of the method is actuating the HVACS. A third step of the method is actuating a windshield washing system of the vehicle such that the solution is dispensed onto the windshield. A fourth step of the method is drawing of the aroma compound into the passenger compartment by the HVACS to freshen the air in the passenger compartment. The present invention is anticipated to be useful in a variety of situations, such as in masking odors in the passenger compartment, helping to improve a mood of an occupant, or the like.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A windshield cleaning composition comprising:
a solution to be added to a wiper fluid reservoir of a vehicle, the solution comprising:
water;
a cleaning agent;
an antifreeze agent; and
an aroma compound, wherein dispensing of the solution onto a windshield of the vehicle positions the aroma compound to be drawn into a passenger compartment of the vehicle by a heating, cooling, and air conditioning system of the vehicle.

2. The windshield cleaning composition of claim 1, wherein the antifreeze agent comprises one or more of ethanol, isopropanol, ethylene glycol, propylene glycol, glycerol, 1,3-butanediol, or 2-butanone.

3. The windshield cleaning composition of claim 1, wherein the antifreeze agent comprises from 0.5 to 50.0% by volume of the solution.

4. The windshield cleaning composition of claim 3, wherein the antifreeze agent comprises from 0.5 to 10.0% by volume of the solution.

5. The windshield cleaning composition of claim 1, wherein the cleaning agent comprises one or more of ammonia, 2-hexoxyethanol, lauryl dimethyl amine oxide, sodium dodecylbenzene sulfonate, isopropanolamine, butoxyethanol, perchloroethylene, tetrachloroethylene, ethanolamine, or sodium hydroxide.

6. The windshield cleaning composition of claim 1, wherein the cleaning agent comprises from 0.2 to 5.0% by volume of the solution.

7. The windshield cleaning composition of claim 6, wherein the cleaning agent comprises from 0.5 to 2.5% by volume of the solution.

8. The windshield cleaning composition of claim 1, wherein the aroma compound comprises from 0.25 to 8.0% by volume of the solution.

9. The windshield cleaning composition of claim 8, wherein the aroma compound comprises from 3.3 to 5.0% by volume of the solution.

10. A windshield cleaning composition comprising a solution to be added to a wiper fluid reservoir of a vehicle, the solution comprising:
water;
a cleaning agent comprising one or more of ammonia, 2-hexoxyethanol, lauryl dimethyl amine oxide, sodium dodecylbenzene sulfonate, isopropanolamine, butoxyethanol, perchloroethylene, tetrachloroethylene, ethanolamine, or sodium hydroxide, the cleaning agent comprising from 0.2 to 5.0% by volume of the solution;
an antifreeze agent comprising one or more of ethanol, isopropanol, ethylene glycol, propylene glycol, glycerol, 1,3 butanediol, or 2-butanone, the antifreeze agent comprising from 0.5 to 50.0% by volume of the solution; and
an aroma compound comprising from 0.25 to 8.0% by volume of the solution, wherein dispensing of the solution onto a windshield of the vehicle positions the aroma compound to be drawn into a passenger compartment of the vehicle by a heating, cooling, and air conditioning system of the vehicle.

11. The windshield cleaning composition of claim 10, wherein:
the cleaning agent comprises from 0.5 to 2.5% by volume of the solution;
the antifreeze agent comprises from 0.5 to 10.0% by volume of the solution; and
the aroma compound comprises from 3.3 to 5.0% by volume of the solution.

12. A method of freshening air in a passenger compartment of a vehicle comprising:
providing a windshield cleaning composition comprising a solution to be added to a wiper fluid reservoir of a vehicle, the solution comprising:
water;
a cleaning agent;
an antifreeze agent; and
an aroma compound, wherein dispensing of the solution onto a windshield of the vehicle positions the aroma compound to be drawn into a passenger compartment of the vehicle by a heating, cooling, and air conditioning system (HVACS) of the vehicle;
adding the solution to the wiper fluid reservoir of the vehicle;
actuating the HVACS;
actuating a windshield washing system of the vehicle such that the solution is dispensed onto the windshield; and
drawing of the aroma compound into the passenger compartment by the HVACS to freshen the air in the passenger compartment.

13. The method claim 12, wherein:
the antifreeze agent comprises from 0.5 to 50.0% by volume of the solution;
the cleaning agent comprises from 0.2 to 5.0% by volume of the solution; and
the aroma compound comprises from 0.25 to 8.0% by volume of the solution.

14. The method claim 13, wherein:
the cleaning agent comprises from 0.5 to 2.5% by volume of the solution;
the antifreeze agent comprises from 0.5 to 10.0% by volume of the solution; and
the aroma compound comprises from 3.3 to 5.0% by volume of the solution.

15. The method of claim 12, wherein the cleaning agent comprises one or more of ammonia, 2-hexoxyethanol, lauryl dimethyl amine oxide, sodium dodecylbenzene sulfonate, isopropanolamine, butoxyethanol, perchloroethylene, tetrachloroethylene, ethanolamine, or sodium hydroxide.

16. The method of claim 12, wherein the antifreeze agent comprises one or more of ethanol, isopropanol, ethylene glycol, propylene glycol, glycerol, 1,3 butanediol, or 2-butanone.

17. The method of claim 12, wherein:
the cleaning agent comprises one or more of ammonia, 2-hexoxyethanol, lauryl dimethyl amine oxide, sodium dodecylbenzene sulfonate, isopropanolamine, butoxyethanol, perchloroethylene, tetrachloroethylene, ethanolamine, or sodium hydroxide, the cleaning agent comprising from 0.2 to 5.0% by volume of the solution;
the antifreeze agent comprises one or more of ethanol, isopropanol, ethylene glycol, propylene glycol, glycerol, 1,3 butanediol, or 2-butanone, the antifreeze agent comprising from 0.5 to 50.0% by volume of the solution; and
the aroma compound comprises from 0.25 to 8.0% by volume of the solution.
